(19) United States
(12) Patent Application Publication
(10) Pub. No.: US 2026/0273008 A1
(43) Pub. Date: Sep. 17, 2026

(54) STRUCTURED LIPID-ANTIOXIDANT-BIOTIC MATRIX FOR FUNCTIONAL FOOD APPLICATIONS WITH DUAL-SITE ORAL AND GASTROINTESTINAL MODULATION

(71) Applicants: Delsys Almeida, Sarasota, FL (US); Guy Suarez Lopez, Sarasota, FL (US)

(72) Inventors: Delsys Almeida, Sarasota, FL (US); Guy Suarez Lopez, Sarasota, FL (US)

(21) Appl. No.: 19/562,529

(22) Filed: Mar. 10, 2026

Related U.S. Application Data
(60) Provisional application No. 63/770,326, filed on Mar. 11, 2025.

(51) Int. Cl. A61K 36/185; A61K 9/00; A61K 31/7004; A61K 36/22; A61K 45/06; A61K 47/46; A61P 1/00

(52) U.S. Cl. CPC A61K 36/5777; A61K 9/0056; A61K 31/7004; A61K 36/22; A61K 45/06; A61K 47/46; A61P 1/00

(57) ABSTRACT

A food composition comprising a lipid phase comprising one or more plant-derived lipids selected from cocoa butter, mango butter, shea butter, cocoa butter equivalents, medium chain triglycerides, and coconut oil. The composition further comprises an antioxidant-rich botanical phase comprising one or more materials selected from cacao-derived materials, pomegranate pomace, carob, and mesquite. The composition further comprises a biotic component comprising one or more of prebiotics, probiotics, postbiotics, and low-glycemic sweeteners. The antioxidant-rich botanical phase and the biotic component are homogeneously dispersed within the lipid phase without microencapsulation. The composition is formulated in a solid and/or non-solid form selected from syrups, gels, pastes, spreads, and direct-to-mouth compositions, and provides antioxidant activity, microbiome modulation, and delivery of bioactive compounds. The composition is configured to provide dual-site functional support along an oral-to-gastrointestinal pathway, modulating the oral microenvironment during oral cavity exposure and supporting gut microbiome composition during gastrointestinal transit.

### CROSS-REFERENCE TO RELATED APPLICATIONS

This application claims the benefit of U.S. Provisional Application No. 63/770,326, entitled “FoodBIOME: A Microbiome-Enhancing Functional Food Matrix for Portion-Control and Bioactive Delivery,” filed Mar. 11, 2025, and U.S. Provisional Application No. 63/998,125, entitled “A Structured Lipid-Antioxidant-Biotic Matrix for Functional Food Applications,” filed Mar. 6, 2026, both of which are hereby incorporated by reference in their entirety.

### FIELD

The present disclosure relates to functional food compositions and delivery systems, and more particularly to both cacao-based and non-cacao based matrices comprising structured lipid, antioxidant, and biotic components formulated in solid or non-solid forms including syrups, gels, pastes, spreads, and direct-to-mouth compositions for dual-site oral health and gastrointestinal microbiome modulation and bioactive delivery.

### BACKGROUND

Cacao-based food products have long been valued for their sensory properties and potential health benefits. Traditional cacao formulations include solid forms such as chocolate bars and tablets, as well as non-solid forms such as syrups, spreads, pastes, and gels. Each physical form presents distinct formulation and processing considerations, particularly when incorporating bioactive ingredients intended to provide functional benefits beyond basic nutrition.

Functional food compositions that deliver multiple bioactive components face technical challenges related to ingredient compatibility, stability, and bioavailability. The increasing awareness of the relationship between diet and health has driven a growing demand for functional foods enriched with bioactive compounds, including polyphenols, carotenoids, vitamins, minerals, peptides, and unsaturated fatty acids, which provide health benefits beyond basic nutrition, including antioxidant, anti-inflammatory, and antimicrobial properties. Lipid-based matrices can serve as carriers for various bioactive compounds, but combining lipids with hydrophilic or amphiphilic ingredients such as antioxidants, prebiotics, and other biotic components introduces complexity due to differences in polarity, solubility, and phase behavior. These challenges are amplified in non-solid forms where the absence of a rigid crystalline structure can reduce physical stability over time.

Antioxidant compounds, including polyphenols derived from cacao and other botanical sources, are susceptible to degradation when exposed to heat, oxygen, light, and moisture. The direct use of bioactive compounds in food matrices is often limited due to challenges related to instability, poor solubility, and insufficient bioavailability. Studies have estimated that only a small fraction of bioactive compounds can be effectively absorbed into the bloodstream, and such low absorption and poor bioavailability often result in failure to reach target tissues in sufficient concentrations. Maintaining antioxidant activity throughout processing, storage, and consumption represents an ongoing challenge in functional food development. Similarly, prebiotic carbohydrates and other biotic components can exhibit tendencies toward crystallization, precipitation, or phase separation when incorporated into lipid-containing matrices, particularly in semi-solid or liquid forms.

The gut microbiome has received increasing attention as a target for dietary interventions aimed at supporting digestive health, metabolic function, and overall well-being. Compositions designed to modulate the oral health and gastrointestinal microbiome can include prebiotics, probiotics, postbiotics, or combinations thereof. Delivering such biotic components in a stable, palatable, and bioavailable form remains an area of active development in the functional food and nutritional supplement industries. Plant-based delivery systems have emerged as a sustainable and efficient approach to overcome numerous challenges associated with the delivery of bioactive compounds, with the aim of improving their stability, bioavailability, and controlled release behaviors.

Maintaining a balanced gut microbiome is crucial for metabolic health, immune function, and overall well-being. Current functional foods and dietary supplements often suffer from low consumer compliance due to pill fatigue, poor bioavailability of bioactive ingredients leading to ineffective nutrient absorption, non-appealing palatability on products offering metabolic benefits, and lack of microbiome-centered support, missing the opportunity to enhance gut health while delivering other functional nutrients.

Existing approaches to incorporating functional ingredients into both cacao and non-cacao-based products have addressed various aspects of these challenges. Some formulations have focused on adding dietary fibers, vitamins, or minerals to conventional chocolate bases. Others have employed encapsulation techniques to protect sensitive bioactive compounds. Lipid-based formulations, including liposomes and solid lipid nanoparticles, have been studied as delivery systems that protect bioactives from degradation, improve solubility, and modulate release kinetics. However, many such systems require complex microencapsulation processes or synthetic encapsulating agents. Approaches that integrate lipid phases, antioxidant-rich botanical materials, and microbiome-modulating biotic components within a unified food matrix suitable for semi-solid or liquid applications remain limited.

Consumer preferences for convenient, portion-controlled delivery forms have driven interest in functional food products that can be consumed directly from packaging or easily incorporated into beverages and other foods. Non-solid forms such as syrups, gels, pastes, and spreads offer versatility in application but present formulation challenges distinct from those encountered in solid chocolate products. Achieving long-term physical and chemical stability while maintaining desirable sensory characteristics in such forms continues to be an area where improvements are sought. There remains a need for functional food compositions that can deliver multiple bioactive components including antioxidants and biotic components in a stable, bioavailable form without requiring complex microencapsulation processes, while providing both oral benefits and gastrointestinal microbiome modulation through a unified matrix architecture.

Current functional foods and nutraceutical delivery systems have generally developed along two separate approaches: oral-care products intended to act locally in the oral cavity, and ingestible products intended to act in the gastrointestinal tract after swallowing. In many cases, existing products and disclosures are single-site in function (oral or gastrointestinal) and/or single-phase in delivery, even when multiple ingredients are included, rather than providing a defined, site-specific sequence of microbiome modulation that includes an oral phase followed by a gastrointestinal phase.

### SUMMARY

This summary is provided to introduce a selection of concepts in a simplified form that are further described below in the detailed description. This summary is a portion of the overall specification and is not intended to identify key features or essential features of the claimed subject matter, nor is it intended to be used as an aid in determining the scope of the claimed subject matter.

According to an aspect of the present disclosure, a microbiome-enhancing food matrix is provided. The microbiome-enhancing food matrix comprises a lipid-based environment selected from cocoa butter, mango butter, shea butter, coconut oil, medium chain triglycerides (MCT) and cocoa butter equivalents. The microbiome-enhancing food matrix further comprises an antioxidant-rich botanical phase comprising one or more botanical materials selected from the group consisting of cacao-derived materials, pomegranate, carob, and mesquite. The microbiome-enhancing food matrix further comprises a microbiome-modulating biotic component comprising one or more of prebiotics, probiotics, postbiotics, and low-glycemic sweeteners. The antioxidant-rich botanical phase and the biotic component are homogeneously dispersed within the lipid-based matrix without microencapsulation and the composition is formulated in a solid or non-solid form selected from the group consisting of syrups, gels, pastes, spreads, and direct-to-mouth compositions.

According to another aspect of the present disclosure, the composition is configured to provide dual-site functional support along an oral-to-gastrointestinal pathway. Upon administration, the composition contacts saliva and one or more oral surfaces during oral cavity exposure, thereby providing an upstream opportunity to modulate the oral microenvironment. The composition is then swallowed and undergoes gastrointestinal transit and delivery, where the structured matrix facilitates delivery of substrates and actives to the gastrointestinal tract and supports downstream modulation of gut microbiome composition.

According to another aspect of the present disclosure, a method for manufacturing a non-solid food composition is provided. The method comprises providing a lipid phase comprising one or more plant-derived lipids selected from the group consisting of cocoa butter, mango butter, shea butter, coconut oil, medium chain triglycerides (MCT) and cocoa butter equivalents. The method further comprises maintaining processing temperatures within a controlled range to prevent degradation of heat-sensitive compounds. The method further comprises homogeneously dispersing an antioxidant-rich botanical phase within the lipid phase, the antioxidant-rich botanical phase comprising one or more antioxidant botanical materials. The method further comprises homogeneously dispersing a biotic component within the lipid phase, the biotic component comprising one or more of prebiotics, probiotics, postbiotics, and low-glycemic sweeteners. The method further comprises reducing particle size of dispersed components to improve mouthfeel and prevent phase separation. The method further comprises forming the composition into pre-measured solid or non-solid unit doses without microencapsulation of the dispersed components.

According to another aspect of the present disclosure, a functional food product is provided. The functional food product comprises a non-solid matrix serving as a delivery system for bioactive compounds, the matrix comprising a lipid phase comprising one or more plant-derived lipids, an antioxidant-rich botanical phase comprising one or more antioxidant botanical materials homogeneously dispersed within the lipid phase, and a biotic component comprising one or more of prebiotics, probiotics, postbiotics, and low-glycemic sweeteners homogeneously dispersed within the lipid phase without microencapsulation, wherein the matrix is formulated in a solid or non-solid form and provides antioxidant activity, microbiome modulation, and delivery of the bioactive compounds.

More specifically the present disclosure provides for food composition, comprising: a lipid phase comprising one or more plant-derived lipids selected from at least one or more of a group consisting of cocoa butter, mango butter, shea butter, cocoa butter equivalents, medium chain triglycerides oil, and coconut oil;
an antioxidant-rich botanical phase comprising at least one or more of a group consisting of cacao-derived materials, pomegranate pomace, carob, and mesquite; and
a biotic component comprising one or more of prebiotics, probiotics, postbiotics, and low-glycemic sweeteners;
wherein said antioxidant-rich botanical phase and said biotic component are homogeneously dispersed within said lipid phase in an absence of microencapsulation, and wherein said composition is formulated in a solid and/or non-solid form selected from a group consisting of syrups, gels, pastes, spreads, and direct-to-mouth compositions.

This can also include cacao-derived materials that comprise one or more of cocoa butter, cocoa liquor, cocoa powder, and cocoa nibs.

In some cases the cacao-derived materials comprise cocoa liquor that provides compounds contributing to antioxidant activity and lipidic phase within the composition.

The low-glycemic sweeteners are selected from at least one or more of a group consisting of tagatose, allulose, xylitol, lactitol, isomalt, date, and inulin.

Also the low-glycemic sweeteners serve as prebiotic substrates for microbial fermentation in a gastrointestinal tract.

In one embodiment the lipid phase functions as a bioactive carrier that provides protection and controlled transport of the antioxidant-rich botanical phase and the biotic component through an oral cavity and into a gastrointestinal tract.

Here the emulsifier may be selected from lecithin derived from at least one of either soy or sunflower.

The composition is packaged in a portion-controlled form selected from one or more of a group consisting of stick-packs, sachets, squeeze pouches, tubes, and single-serve packets.

In another embodiment, the one or more optional bioactive additives are selected from at least one or more of a group consisting of berberine, nootropics, carotenoids, and adaptogens dispersed within the lipid phase.

In yet another embodiment one or more medical food components selected from a group consisting of: gut health agents comprising one or more of dietary fibers, prebiotic substrates, enzyme preparations, or postbiotic preparations; plant proteins comprising one or more of plant protein isolates, plant protein concentrates, or hydrolyzed plant proteins; iron deficiency addressing components comprising one or more of an iron source and an absorption cofactor; and oral wellness components comprising one or more of polyols, buffering agents, remineralization agents, zinc-containing salts, or oral probiotic/postbiotic preparations.

The biotic component comprises prebiotic carbohydrates configured to support production of short-chain fatty acids through microbial fermentation in a gastrointestinal tract.

Here the composition is configured to provide dual-site functional support along an oral-to-gastrointestinal pathway, wherein upon administration the composition contacts saliva and one or more oral surfaces during oral cavity exposure that provides modulation of an oral microenvironment, and wherein the composition subsequently undergoes gastrointestinal transit to support modulation of gut microbiome composition.

The pomegranate pomace serves dual functions within the composition, providing both antioxidant protection to the lipid phase and physical scaffolding for the biotic component.

The lipid phase may comprise a combination of cocoa butter and mango butter in a ratio that is specifically configured to enable high loading of the biotic component and the pomegranate pomace and allows stabilization of crystallization in solid forms and/or causes phase separation in non-solid forms.

In another embodiment the functional food product comprises:
a matrix that is a delivery system for bioactive compounds, the matrix comprising:
a lipid phase comprising one or more plant-derived lipids;
an antioxidant-rich botanical phase comprising one or more botanical materials homogeneously dispersed within said lipid phase; and
a biotic component comprising one or more of prebiotics, probiotics, postbiotics, and low-glycemic sweeteners homogeneously dispersed within the lipid phase in an absence of microencapsulation;
wherein the matrix is formulated in a solid and/or non-solid form and provides antioxidant activity, microbiome modulation, oral wellness support, and delivery of the bioactive compounds.

Here the plant-derived lipids are selected from at least one or more of a group consisting of cocoa butter, mango butter, shea butter, cocoa butter equivalents, medium chain triglycerides oil, and coconut oil.

In this case the botanical materials are selected from at least one or more of a group consisting of cacao-derived materials, pomegranate pomace, carob, and mesquite.

Importantly the solid and/or non-solid forms of the product and composition are selected from one or more of a group consisting of syrups, gels, pastes, spreads, and direct-to-mouth compositions.

Here the lipid phase comprises cocoa butter in a proportion of at least 20% by weight of the total lipid phase.

In addition, the antioxidant-rich botanical phase comprises a combination of cocoa liquor and pomegranate pomace, and further comprises carob and/or mesquite.

Here the biotic component comprises a combination of allulose as a prebiotic and one or more probiotic microorganisms.

The food matrix exhibits a viscosity that enables dispensing from a squeeze pouch or tube at room temperature.

The functional food product of claim 15, wherein the matrix maintains physical stability in an absence of phase separation for at least six months at room temperature storage conditions.

For this functional food product the matrix is configured to function as a biphasic delivery system that releases a first bioactive portion comprising tagatose and polyphenols within an oral cavity during consumption and protects a second bioactive portion comprising the biotic component during gastric transit for colonic delivery.

In another embodiment a portion-controlled delivery system for bioactive compounds comprises:
a sealed package that contains a pre-measured quantity of a matrix composition, the composition comprising:
a lipid phase comprising one or more plant-derived lipids selected from at least one or more of a group consisting of cocoa butter, mango butter, shea butter, cocoa butter equivalents, medium chain triglycerides oil and coconut oil;
an antioxidant-rich botanical phase comprising one or more of a group consisting of cacao-derived materials, pomegranate pomace, carob, and mesquite homogeneously dispersed within the lipid phase; and
a biotic component comprising one or more of prebiotics, probiotics, postbiotics, and low-glycemic sweeteners homogeneously dispersed within the lipid phase in an absence of microencapsulation;
wherein the sealed package is configured for direct dispensing of the composition into an oral cavity or onto food products.

This portion-controlled delivery system may utilize a sealed package that is a stick-pack.

In another embodiment the sealed package is a squeeze pouch.

This composition further comprises one or more natural sweeteners selected from one or more of a group consisting of monk fruit extract and stevia extract.

The portion-controlled delivery system and composition further comprises one or more flavors selected from one or more of a group consisting of vanilla, mint, caramel, caramel cream, fruit, floral, citrus, and berry flavors, wherein the composition is formulated as a clean-label product that does not include synthetic encapsulating agents.

In another embodiment a form-agnostic food matrix composition, comprising:
a lipid phase comprising one or more plant-derived lipids;
an antioxidant-rich botanical phase comprising at least one or more of a group consisting of cacao-derived materials, pomegranate pomace, carob, and mesquite homogeneously dispersed within the lipid phase; and;
a biotic component comprising one or more of prebiotics, probiotics, postbiotics, and low-glycemic sweeteners homogeneously dispersed within the lipid phase in an absence of microencapsulation;
wherein the pomegranate pomace fibers dispersed within the lipid phase act as a physical scaffold that maintains matrix integrity in both solid and/or non-solid states, and wherein the composition maintains antioxidant and biotic viability regardless of physical form.

### BRIEF DESCRIPTION OF THE DRAWINGS

Non-limiting and non-exhaustive examples are described with reference to the following figures.

FIG. 1 illustrates a diagram 100 depicting the synergistic gut health benefits achieved through the combination of three component categories for gut microbiome modulation, including an antioxidants category 102 comprising cocoa, pomegranate, and other botanical materials, a lipids category 104 comprising cocoa butter, mango butter, shea butter, and other plant-derived lipids, and a biotics category 106 comprising prebiotics, probiotics, and postbiotics, with a gut illustration 108 at the intersection of the three categories indicating the synergistic effect of the combined components on the gut microbiome 110.

FIG. 2 illustrates an applications diagram 200 showing the food matrix platform connecting to functional food outputs 202.

FIG. 3 illustrates the Food Matrix platform concept 300 comprising a Platform Stack 302 with four stacked elements including an indulgent carrier 304, a functional matrix 306, a bioactive payload 308, and oral plus gut delivery 310.

FIG. 4 illustrates a platform definition diagram 400 for the structured lipid-antioxidant-biotic composition, characterizing the composition as a food-as-medicine composition that delivers intrinsic, function-first microbiome support and serves as a delivery system for health-outcome-specific bioactive compounds, with three component categories shown as separate panels comprising lipids 402, Antioxidants 404, and Biotics 406.

FIG. 5 illustrates a modular bioactive toolkit 500 comprising six component categories including Biotics 502 (prebiotics, probiotics, and postbiotics), Polyphenols 504 (cocoa flavanols, fruit and plant extracts, and pomace ingredients), Fibers and substrates 506 (fermentable fibers, resistant starches, and SCFA precursors), lipids 508 (cocoa and plant fats, emulsifier systems, and texture plus stability components), Minerals and co-factors 510 (electrolytes, trace minerals, and synergists), and Sweetener systems 512 (low-GI options, taste balancing components, and consumer acceptance considerations).

FIG. 6 illustrates an application map 600 showing the Food Matrix Platform 602 connecting to five application verticals including Functional foods and beverages 604, Nutritional supplements 606, Medical and clinical nutrition 608, Oral health and dental nutrition 610, and Personalized nutrition and longevity 612.

FIG. 7 illustrates a validation roadmap diagram 700 showing four sequential stages comprising In-vitro screening 702 (gut modulation assays and compatibility checks), Pilot studies 704 (directional signals and biomarker selection), Controlled validation 706 (dose-response and clinical endpoints), and Scale plus extensions 708 (new payloads and new verticals).

FIG. 8 illustrates a platform summary diagram 800 organized into three main functional categories comprising Platform 802 (configurable matrix stack with carrier, scaffold, payload, and route), Delivery 804 (portion control with indulgent compliance, oral delivery, and gut function-first programs), and Validation 806 (pilot signals with roadmap to controlled substantiation).

FIG. 9 illustrates an example oral administration pathway 900 for a structured lipid-antioxidant-biotics food matrix composition and corresponding dual-site functional support. A composition 904 is orally administered and exposed within the oral cavity 906 through contact with saliva and one or more oral surfaces, followed by gastrointestinal transit and delivery 910. The pathway may produce functional support outcomes including modulation of the oral microenvironment 908 and modulation of gut microbiome composition 912, consistent with sequential oral-to-gastrointestinal functional support.

FIG. 10 illustrates example dual-site modulation signals 1000 associated with daily oral administration of a food matrix composition comprising structured lipids, antioxidants, and biotics 1002. In example embodiments, administration of the composition is associated with gut wellness readouts 1004 including taxa signals indicating a shift in mycobiome and bacteriome balance and a decrease in stool pH consistent with fermentation-related signals, and with oral wellness readouts 1006 including a decreased cariogenic bacteria signal and downward shifts in saliva marker signals.

FIG. 11 illustrates oral wellness signals 1100 measured from saliva samples collected at baseline and at follow-up after daily administration of a structured lipid-antioxidant-biotics food matrix composition for approximately 30 days. The left panel 1102 summarizes the direction-of-change in saliva parameters as counts of subjects exhibiting decreases or increases for cariogenic bacteria, protein, acidity, and leukocyte signals. The right panel 1104 provides wellness-associated interpretations including reduced cariogenic pressure 1106, reduced inflammatory signal 1108, reduced acid-stress environment 1110, and reduced inflammatory activity 1112.

FIG. 12 illustrates gut wellness signals 1200 associated with daily administration of a structured lipid-antioxidant-biotics food matrix composition. Microbiome modulation signals 1202 are summarized as directional taxa signals comprising dysbiosis-linked taxa signals 1204 indicating reduced dysbiosis pressure 1206, beneficial taxa signals 1208 indicating community support 1210, and a stool pH signal 1212 indicating SCFA-related signals 1214.

FIG. 13 illustrates example embodiments and application forms 1300 of a food matrix composition comprising structured lipids, antioxidants, and biotics 1302 having inherent dual-site signals. The composition may be formulated with added bioactives 1304 as nutritional supplements, medical foods, and functional nutrition products, with non-limiting bioactive examples 1306 including CoQ10, nootropics, zeaxanthin and lutein, iron, and ashwagandha. Alternatively, the composition may include food additives, flavors, spices, and inclusions 1308 and be configured as functional foods and beverages.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

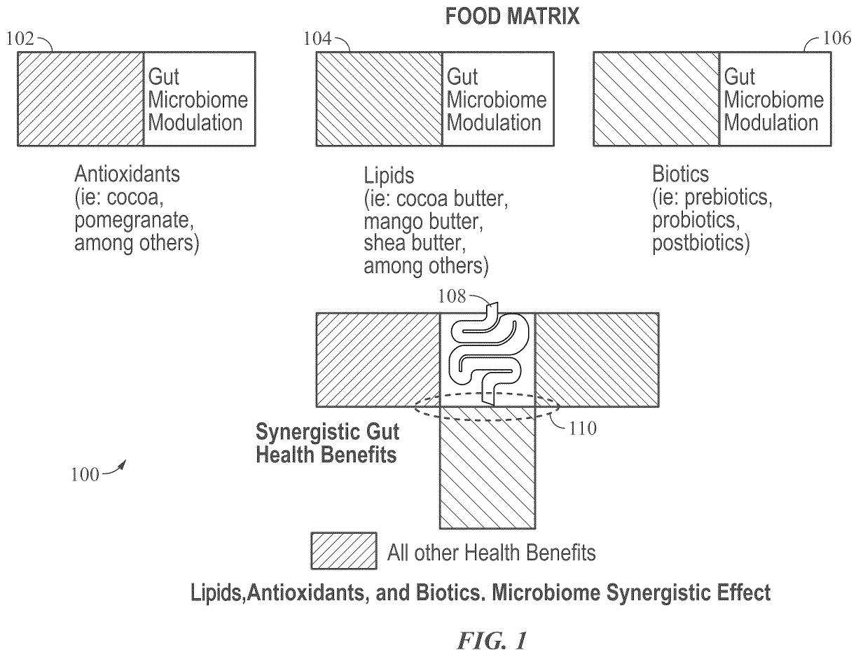

The present disclosure relates to a composition comprising a structured lipid phase, an antioxidant-rich botanical phase, and one or more biotic components. As illustrated in FIG. 1, the diagram 100 shows the composition that achieves synergistic gut health benefits through the combination of three component categories: an antioxidants category 102, a lipids category 104, and biotics category 106, with a gut illustration 108 at the intersection indicating the synergistic effect 110. The composition can be formulated in solid or non-solid forms including syrups, gels, pastes, spreads, and direct-to-mouth compositions. The structured lipid phase can comprise one or more of cocoa butter, mango butter, shea butter, medium chain triglycerides, coconut oil, and other plant-derived lipids. The antioxidant-rich botanical phase can comprise cacao-derived materials, pomegranate, carob, mesquite, and other fruit and legume materials. The biotic components can comprise prebiotics, probiotics, postbiotics, and low-glycemic sweeteners.

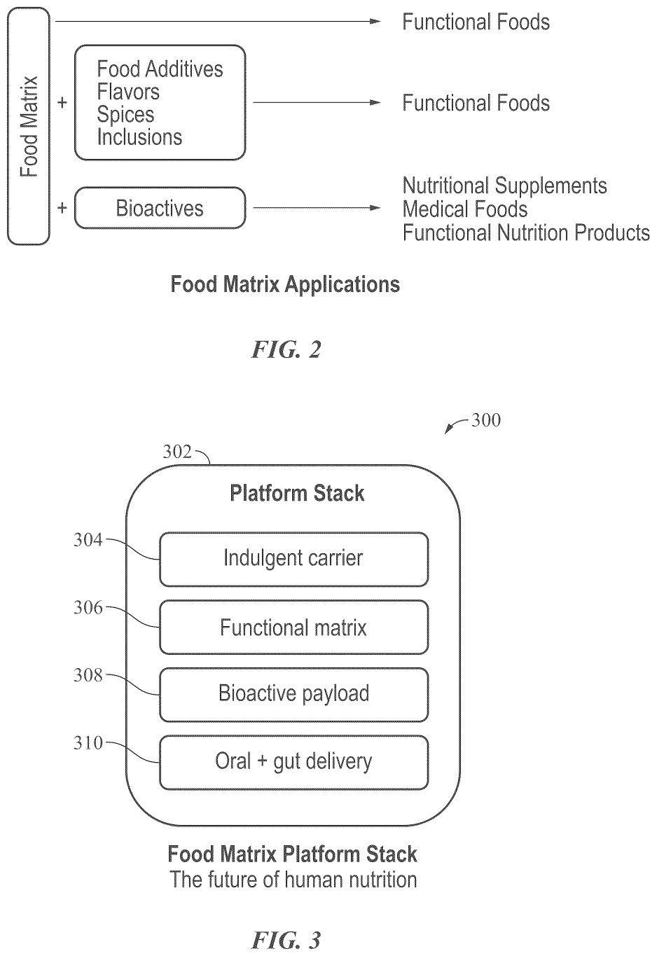

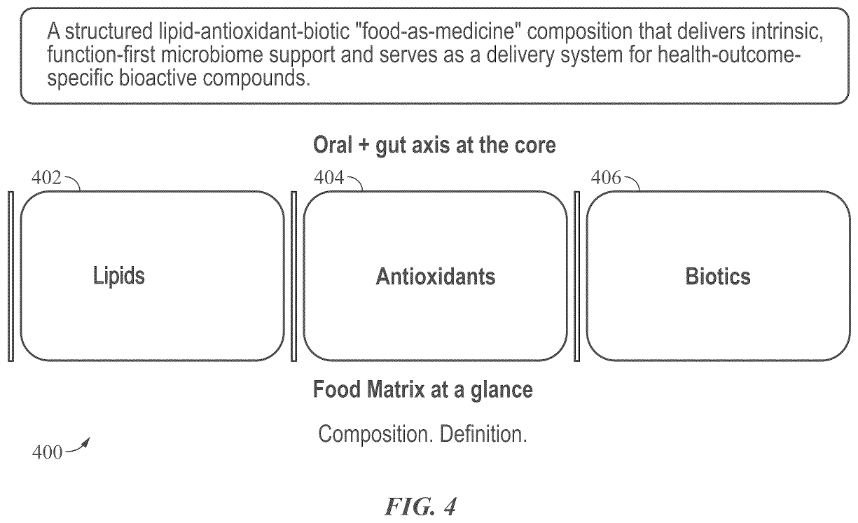

The composition forms part of a unified platform that encompasses both solid embodiments and non-solid forms, as illustrated in FIG. 2 and FIG. 3. Referring to FIG. 2, the applications diagram 200 shows the food matrix platform connecting to functional food outputs 202. Referring to FIG. 3, the Food Matrix platform concept 300 comprises a Platform Stack 302 with four stacked elements including an indulgent carrier 304, a functional matrix 306, a bioactive payload 308, and oral plus gut delivery 310. Referring to FIG. 4, the platform definition diagram 400 shows three component categories comprising Lipids 402, Antioxidants 404, and Biotics 406. The solid embodiments can be produced via controlled crystallization of cacao lipids, while the non-solid forms can exist as semi-solid or liquid physical states. Both the solid embodiments and the non-solid forms represent alternative physical forms of the same structured composition. The unified platform maintains a core architecture comprising the lipid phase, the antioxidant-rich botanical phase, and the biotic components across the different physical forms.

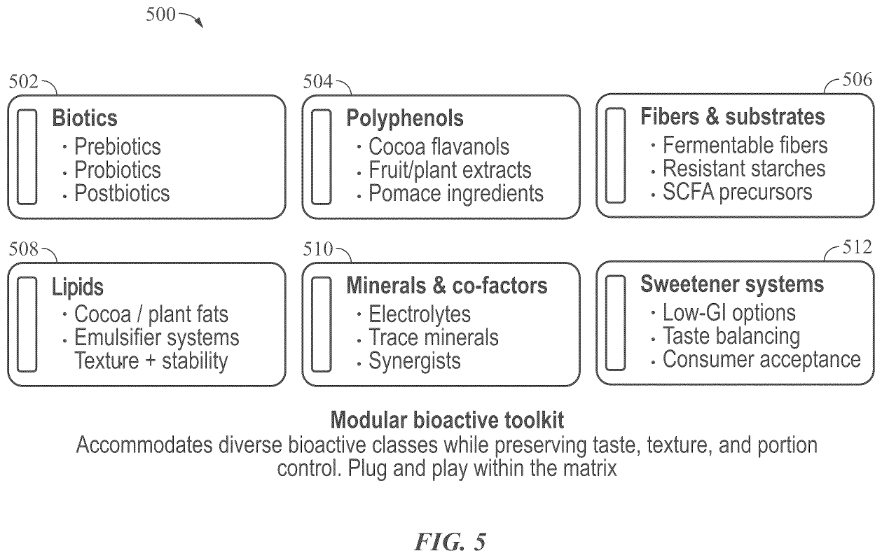

Referring to FIG. 5, the modular bioactive toolkit 500 comprises six component categories including Biotics 502, Polyphenols 504, Fibers and substrates 506, Lipids 508, Minerals and co-factors 510, and Sweetener systems 512. This modular architecture demonstrates how the various functional components can be combined and configured within the lipid-based matrix to achieve different functional food formulations while maintaining the core structural and delivery characteristics of the composition.

The composition can achieve bioavailability enhancement without microencapsulation. The bioavailability enhancement can be achieved by relying on lipid-based composition dispersion. The lipid phase can function as a bioactive carrier, providing stability, enhancing absorption, and protecting bioactive compounds dispersed within the lipid phase. The antioxidant-rich botanical phase and the biotic components can be homogeneously dispersed within the lipid phase. The dispersion of the antioxidant-rich botanical phase and the biotic components within the lipid phase can eliminate encapsulation requirements while enabling effective delivery of the dispersed components.

The lipid composition can include cocoa butter, mango butter, shea butter, medium chain triglycerides, coconut oil, cocoa butter equivalents, and other plant-derived lipids. Cocoa butter can serve as a primary lipid component in the structured matrix. Mango butter can be included in the lipid composition to contribute to the lipid phase characteristics. Shea butter can be incorporated as a plant-derived lipid component. Coconut oil can be included as a plant-derived lipid component in the structured matrix. Medium chain triglycerides can be included as a plant-derived lipid component in the structured matrix. Cocoa butter equivalents can be included in the lipid composition to contribute to the lipid phase characteristics. The plant-derived lipids can be combined in various proportions to achieve desired physical and functional properties of the lipid phase.

The lipid phase can function as a bioactive carrier within the composition. The lipid phase can provide stability to bioactive compounds dispersed within the lipid phase. The lipid phase can protect bioactives from degradation during storage and processing. The lipid phase can provide protection and controlled transport of antioxidants and biotic components. The antioxidant components can be protected from oxidation by the lipid phase. The biotic components can be protected from environmental factors by the lipid phase. The controlled transport of the antioxidant and biotic components can facilitate delivery of the polyphenolic and biotic components through the oral cavity and into the gastrointestinal tract.

The antioxidant-rich botanical composition can include cacao-derived materials, pomegranate pomace, carob, mesquite, and other fruit and legume materials. Cacao-derived materials can include cocoa liquor, cocoa powder, and cocoa nibs. The cacao-derived materials can provide compounds that contribute to antioxidant activity within the composition. Pomegranate pomace can be included as a botanical material that provides antioxidant compounds. The pomegranate pomace can contribute to the antioxidant activity of the composition and can support microbiome health modulation.

The pomegranate pomace can serve dual functions within the composition, providing both antioxidant protection to the lipid phase and physical scaffolding for biotic components. The pomegranate pomace can comprise upcycled fruit pomace that serves as a solid carrier and prebiotic substrate within the lipid matrix. The solid particulate nature of the pomegranate pomace can distinguish the composition from prior art approaches that use pomegranate extracts in liquid or paste form rather than solid pomace with structural function. The pomegranate pomace particles can serve as a solid carrier for biotic components, protecting the biotic components from moisture and oxidation within the lipid matrix. The fibrous structure of the pomegranate pomace can provide a physical microenvironment that contributes to the stability of the dispersed biotic components.

The microbiome-modulating biotic components can comprise prebiotics, probiotics, postbiotics, and low-glycemic sweeteners. The biotic components can be homogeneously dispersed within the lipid phase of the composition. The dispersion of the biotic components within the lipid phase can provide protection from environmental factors during storage and processing. Prebiotics can comprise fermentable carbohydrates that serve as substrates for microbial metabolism in the gastrointestinal tract. The prebiotics can include inulin and other prebiotic fibers. Probiotics can comprise live microorganisms that can confer health benefits when consumed. Postbiotics can comprise metabolic byproducts or cellular components derived from probiotic microorganisms. Low-glycemic sweeteners can comprise tagatose, allulose, xylitol, lactitol, isomalt, date or inulin.

The combination of tagatose with pomegranate polyphenols and other biotic components within the composition can create a synbiotic effect. Tagatose can function as a known prebiotic that provides fermentable substrate for beneficial microbial populations. The pomegranate polyphenols from the pomegranate pomace can have prebiotic potential through their interaction with microbial communities. The combination of tagatose and pomegranate pomace within the lipid matrix can provide synergistic prebiotic effects that extend beyond the additive benefits of the individual components. The synbiotic effect can support both oral microbiome modulation during the oral phase of consumption and gastrointestinal microbiome modulation during the gut phase following ingestion.

The composition can include lecithin as an optional emulsifying component. Lecithin can be derived from soy, sunflower, or other plant sources. The composition can include natural sweeteners as optional components for taste modification, including monk fruit extract, stevia extract, coconut sugar, date sugar, and maple syrup. The composition can include flavors as optional components for sensory enhancement, including vanilla, mint, caramel, caramel cream, fruit, floral, citrus, berry, and other fruit flavors. The composition can include inclusions as optional components for sensory enhancement, including nut pieces, dried fruit pieces, dried spice pieces, seed pieces, and crisped grain pieces.

The composition can include berberine as an optional bioactive additive. Berberine can function as a GLP-1 modulator within the composition. The composition can include nootropics as optional bioactive additives for cognitive enhancement. The composition can include carotenoids as optional bioactive additives, including xanthophyll carotenoids such as zeaxanthin and lutein. The composition can include adaptogens as optional bioactive additives that support the body's response to stress, such as ashwagandha.

The composition can include gut health agents as medical food components, including glutamine, zinc carnosine, butyrate, and other compounds that support gut epithelial health. The composition can include plant proteins as medical food components, including pea protein, hemp protein, rice protein, and other plant-derived protein sources. The composition can include iron deficiency addressing components as medical food additives, including ferrous bisglycinate, iron polysaccharide complex, and vitamin C. The composition can include oral wellness components as medical food additives, including xylitol, hydroxyapatite, and other compounds that support dental health.

The composition can be formulated in semi-solid and liquid forms including syrups, pastes, gels, and spreads. The syrup form can comprise a liquid or pourable semi-liquid consistency suitable for direct consumption or incorporation into beverages. The paste form can comprise a semi-solid consistency suitable for dispensing from tubes, pouches, or stick-packs. The gel form can comprise a semi-solid consistency with gel-like rheological properties suitable for direct oral consumption. The spread form can comprise a semi-solid consistency suitable for spreading onto bread, crackers, fruit, or other food substrates.

The composition can be delivered in a stick-pack form for portion-controlled consumption. The stick-pack form can comprise an elongated, sealed pouch containing a pre-measured quantity of the composition. The pre-measured quantity in the stick-pack form can enable consistent dosing of the bioactive compounds contained within the composition. The composition can be designed to address pill fatigue experienced by consumers of dietary supplements by providing an alternative delivery form that does not require swallowing of solid dosage forms.

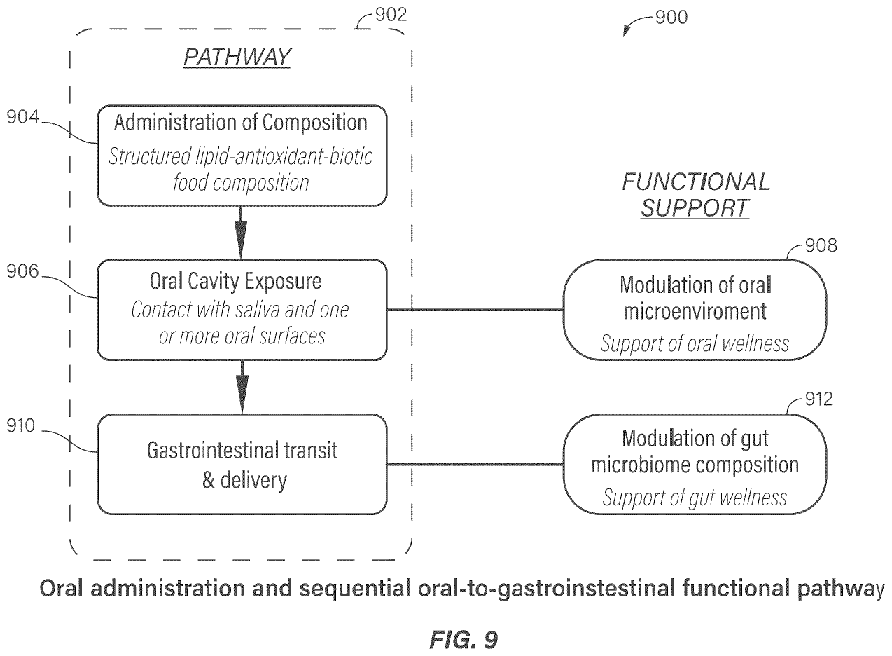

As illustrated in FIG. 9, the composition is configured to provide dual-site functional support along an oral-to-gastrointestinal pathway 900. Upon administration of the composition 904, the composition contacts saliva and one or more oral surfaces during oral cavity exposure 906, thereby providing an upstream opportunity to modulate the oral microenvironment 908. The composition is then swallowed and undergoes gastrointestinal transit and delivery 910, where the structured matrix facilitates delivery of substrates and actives to the gastrointestinal tract and supports downstream modulation of gut microbiome composition 912.

The composition can function as a biphasic or sequential bio-delivery system configured to perform distinct biological functions based on digestion phase. In a first phase occurring in the oral cavity, the lipid matrix can melt during consumption, releasing tagatose and antioxidants from the antioxidant-rich botanical phase to interact with oral biofilms, modulate oral pH, inflammatory processes, and influence oral microbial communities. In a second phase occurring in the gastrointestinal tract, the pomegranate pomace fibers and lipid structure can protect biotic components during gastric transit for colonic delivery. The biphasic delivery approach can enable dual-site efficacy, treating both the oral cavity as an upstream entryway and the gastrointestinal tract as a downstream destination with a single unit dose of the composition.

The lipid matrix can increase residence time of tagatose and polyphenols on dental biofilms through mucoadhesive properties of the lipid phase. The lipid-mediated architecture can promote consistent oral exposure during consumption by reducing immediate washout of solids by saliva. Medium chain triglycerides (MCT oils), when included in the lipid phase, can have unique solvent properties that help polyphenols penetrate plaque layers in the mouth.

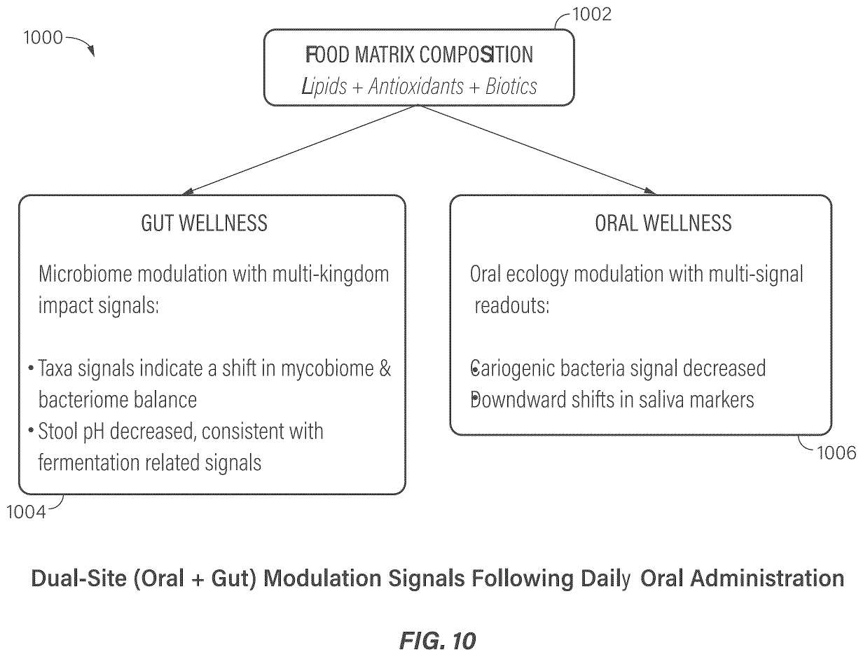

As illustrated in FIG. 10, the dual-site modulation signals 1000 associated with daily oral administration of the food matrix composition 1002 include gut wellness readouts 1004 and oral wellness readouts 1006. The gut wellness readouts can include taxa signals indicating a shift in mycobiome and bacteriome balance and a decrease in stool pH consistent with fermentation-related signals. The oral wellness readouts can include a decreased cariogenic bacteria signal and downward shifts in saliva marker signals.

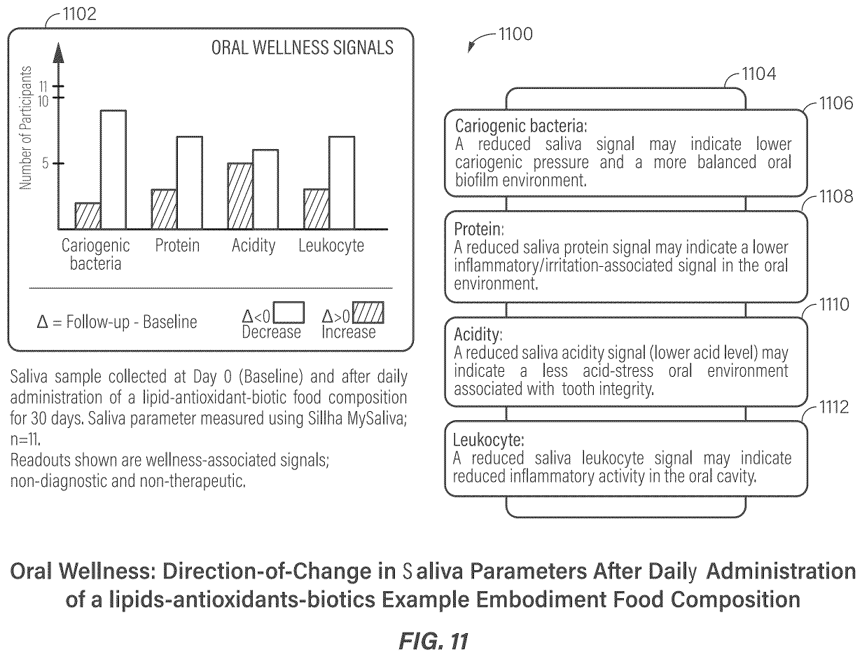

As illustrated in FIG. 11, oral wellness signals 1100 can be measured from saliva samples collected at baseline and at follow-up after daily administration of the composition. The direction-of-change panel 1102 summarizes changes in saliva parameters including cariogenic bacteria, protein, acidity, and leukocyte signals. The interpretive panel 1104 indicates that reduced cariogenic bacteria signal 1106 may indicate lower cariogenic pressure, reduced protein signal 1108 may indicate lower inflammatory signal, reduced acidity signal 1110 may indicate less acid-stress environment, and reduced leukocyte signal 1112 may indicate reduced inflammatory activity.

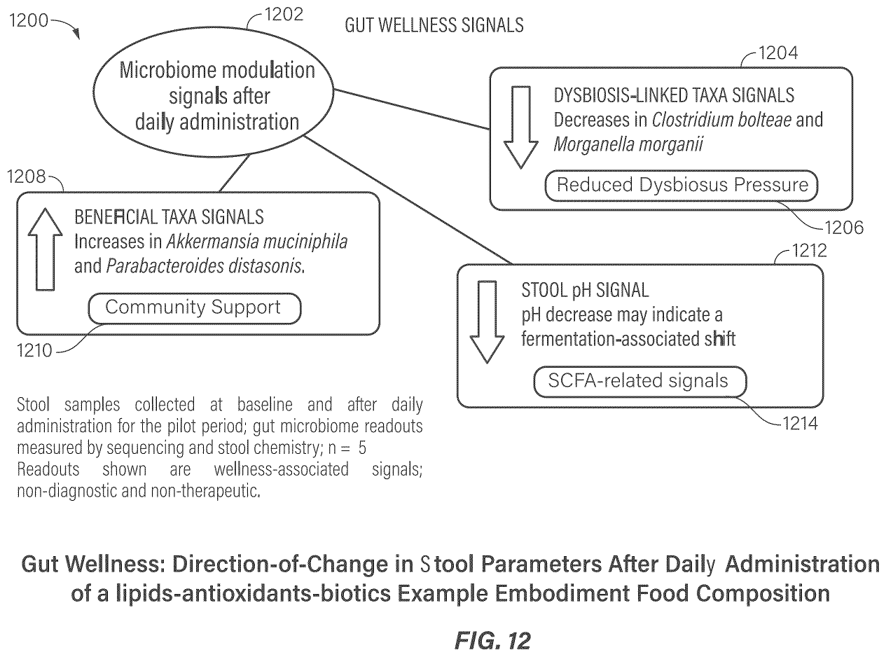

As illustrated in FIG. 12, gut wellness signals 1200 can include microbiome modulation signals 1202 comprising dysbiosis-linked taxa signals 1204 showing decreases in organisms such as Clostridium bolteae and Morganella morganii indicating reduced dysbiosis pressure 1206, beneficial taxa signals 1208 showing increases in organisms such as Akkermansia muciniphila and Parabacteroides distasonis indicating community support 1210, and stool pH signal 1212 indicating fermentation-associated shift consistent with SCFA-related signals 1214.

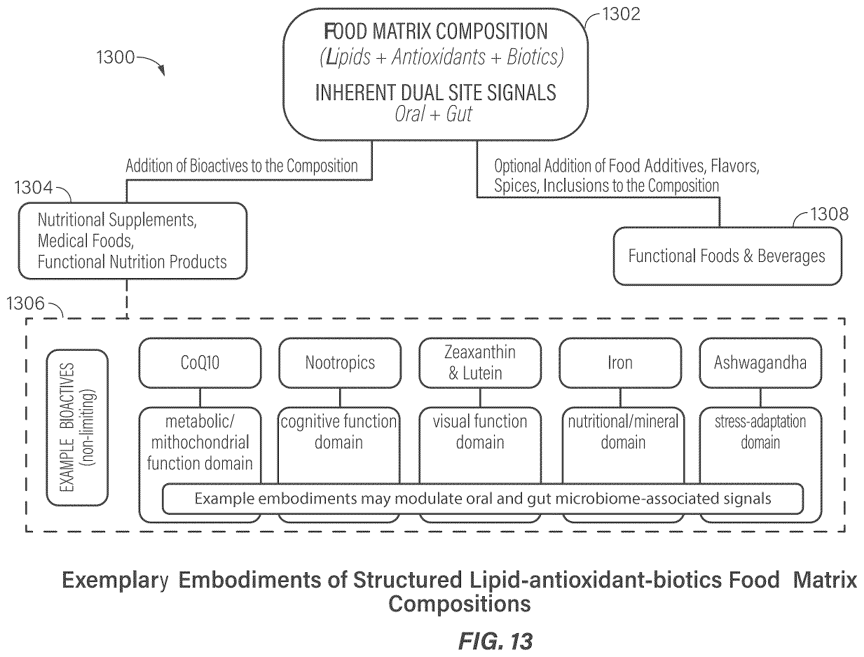

As illustrated in FIG. 13, the example embodiments and application forms 1300 show the food matrix composition 1302 with inherent dual-site signals can be formulated with added bioactives 1304 as nutritional supplements, medical foods, and functional nutrition products. Non-limiting bioactive examples 1306 include CoQ10 for metabolic and mitochondrial function, nootropics for cognitive function, zeaxanthin and lutein for visual function, iron for nutritional and mineral support, and ashwagandha for stress-adaptation. Alternatively, the composition may include food additives, flavors, spices, and inclusions 1308 and be configured as functional foods and beverages.

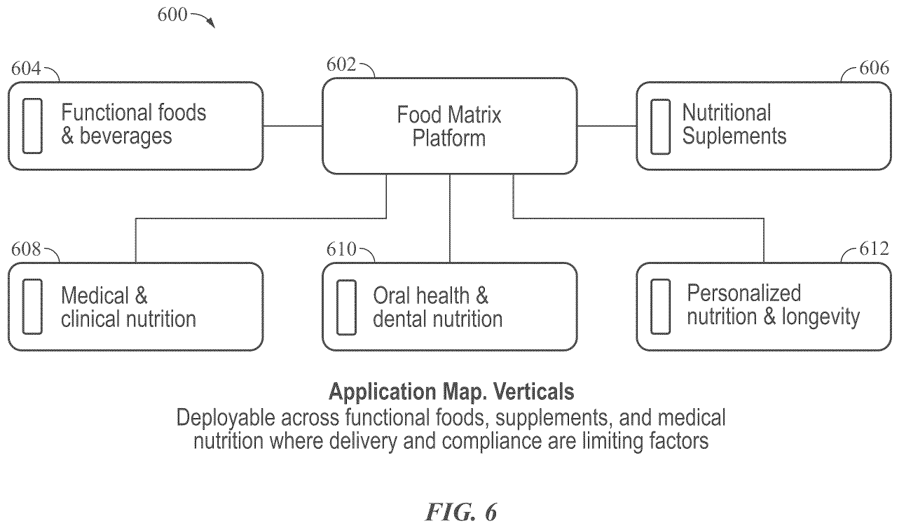

Referring to FIG. 6, the application map 600 shows the Food Matrix Platform 602 connecting to five application verticals including Functional foods and beverages 604, Nutritional supplements 606, Medical and clinical nutrition 608, Oral health and dental nutrition 610, and Personalized nutrition and longevity 612. The composition demonstrates versatility across these different product categories. The structured lipid-antioxidant-biotic matrix can maintain the core architecture comprising the lipid phase, the antioxidant-rich botanical phase, and the biotic phase components across the different product applications.

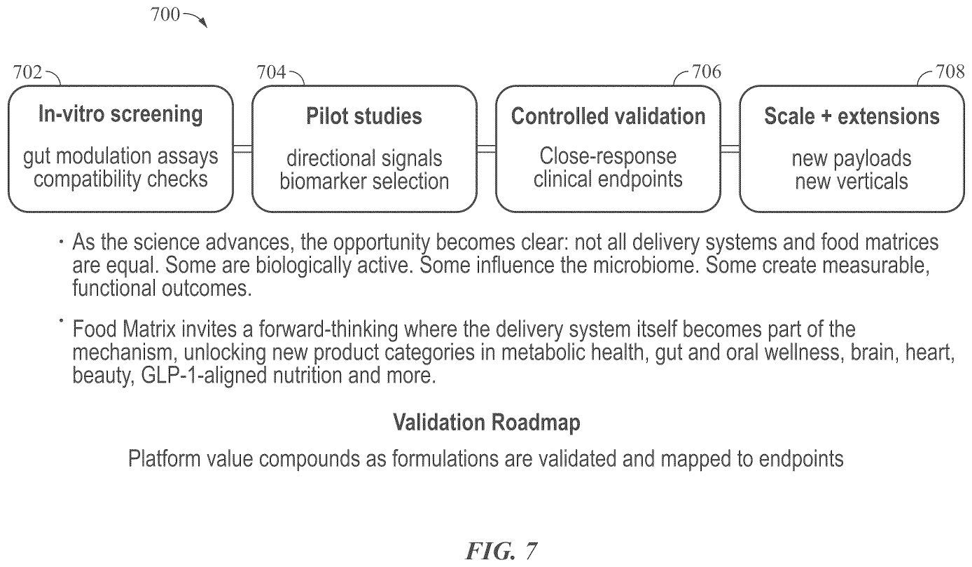

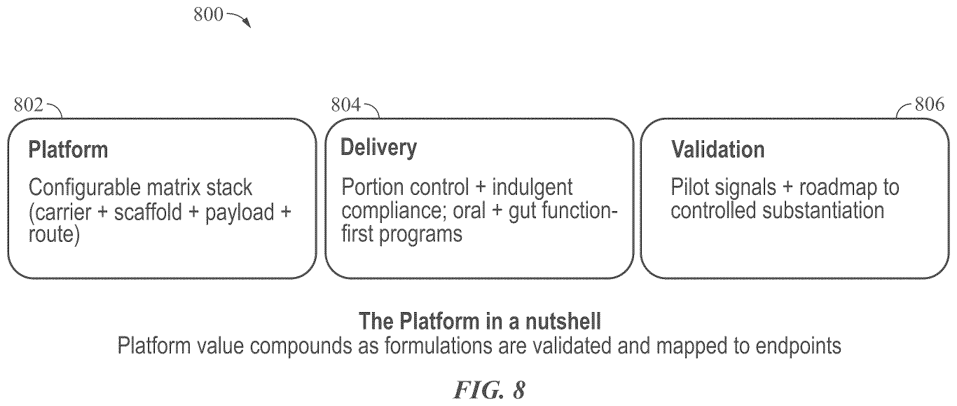

Referring to FIG. 7, the validation roadmap diagram 700 shows four sequential stages comprising In-vitro screening 702, Pilot studies 704, Controlled validation 706, and Scale plus extensions 708. Referring to FIG. 8, the platform summary diagram 800 shows the three main functional categories comprising Platform 802, Delivery 804, and Validation 806.

The manufacturing process for the composition can include maintaining processing temperatures within a controlled range to prevent degradation of heat-sensitive compounds. The manufacturing process can include homogeneously dispersing the biotic components within the lipid phase through mixing operations conducted under controlled temperature conditions. The manufacturing process can include homogeneously dispersing the antioxidant-rich botanical phase within the lipid phase. The manufacturing process can include reducing particle size of the dispersed components to improve mouthfeel and prevent phase separation. The manufacturing process can include forming the matrix into pre-measured solid or non-solid unit doses by filling stick-packs, squeeze pouches, tubes, single-serve packets, or other portion-controlled packaging forms.

The composition can act as a form-agnostic universal lipid matrix that maintains antioxidant and biotic viability in both solid and non-solid states. The pomegranate pomace fibers dispersed within the lipid phase can act as a physical scaffold that maintains matrix integrity in both solid or non-solid states. The form-agnostic nature of the composition can enable the same clinical efficacy and bioactive survival regardless of the final product form. The dual-axis function-first utility of the composition can be preserved whether the composition is administered as a solid embodiment or a flowable embodiment, as the structured lipid-antioxidant-biotic architecture remains consistent across physical forms.

### Working Example 1:

One example by weight percentages (or volume percent for liquid products) of a composition that is an embodiment of the present disclosure includes the following;

The lipid phase includes the following components with the following ranges;
Cocoa butter: 20-35%
   Mango butter: 1-5%
For the Antioxidant phase:
   Total cocoa solids: 20-40%
pomegranate pomace: 1.5-3.0%
and for the Biotic phase:
   Tagatose: 32-40%
Suggested Emulsifier:
   Sunflower Lecithin: 0.5-2.0%

A number of implementations have been described. Nevertheless, it will be understood that various modifications can be made without departing from the spirit and scope of the disclosure. Accordingly, other implementations are within the scope of the following claims.

We claim:

1. A food composition, comprising: a lipid phase comprising one or more plant-derived lipids selected from at least one or more of a group consisting of cocoa butter, mango butter, shea butter, cocoa butter equivalents, medium chain triglycerides oil, and coconut oil;
an antioxidant-rich botanical phase comprising at least one or more of a group consisting of cacao-derived materials, pomegranate pomace, carob, and mesquite; and
a biotic component comprising one or more of prebiotics, probiotics, postbiotics, and low-glycemic sweeteners;
wherein said antioxidant-rich botanical phase and said biotic component are homogeneously dispersed within said lipid phase in an absence of microencapsulation, and wherein said composition is formulated in a solid and/or non-solid form selected from a group consisting of syrups, gels, pastes, spreads, and direct-to-mouth compositions.

2. The composition of claim 1, wherein said cacao-derived materials that comprise one or more of cocoa butter, cocoa liquor, cocoa powder, and cocoa nibs.

3. The composition of claim 2, wherein said cacao-derived materials comprise cocoa liquor that provides compounds contributing to antioxidant activity and lipidic phase within said composition.

4. The composition of claim 1, wherein said low-glycemic sweeteners are selected from at least one or more of a group consisting of tagatose, allulose, xylitol, lactitol, isomalt, date, and inulin.

5. The composition of claim 4, wherein said low-glycemic sweeteners serve as prebiotic substrates for microbial fermentation in a gastrointestinal tract.

6. The composition of claim 1, wherein said lipid phase functions as a bioactive carrier that provides protection and controlled transport of said antioxidant-rich botanical phase and said biotic component through an oral cavity and into a gastrointestinal tract.

7. The composition of claim 1, further comprising an emulsifier selected from lecithin derived from at least one of either soy or sunflower.

8. The composition of claim 1, wherein said composition is packaged in a portion-controlled form selected from one or more of a group consisting of stick-packs, sachets, squeeze pouches, tubes, and single-serve packets.

9. The composition of claim 1, further comprising one or more optional bioactive additives selected from at least one or more of a group consisting of berberine, nootropics, carotenoids, and adaptogens dispersed within said lipid phase.

10. The composition of claim 1, further comprising one or more medical food components selected from a group consisting of: gut health agents comprising one or more of dietary fibers, prebiotic substrates, enzyme preparations, or postbiotic preparations; plant proteins comprising one or more of plant protein isolates, plant protein concentrates, or hydrolyzed plant proteins; iron deficiency addressing components comprising one or more of an iron source and an absorption cofactor; and oral wellness components comprising one or more of polyols, buffering agents, remineralization agents, zinc-containing salts, or oral probiotic/postbiotic preparations.

11. The composition of claim 1, wherein said biotic component comprises prebiotic carbohydrates configured to support production of short-chain fatty acids through microbial fermentation in a gastrointestinal tract.

12. The composition of claim 1, wherein said composition is configured to provide dual-site functional support along an oral-to-gastrointestinal pathway, wherein upon administration said composition contacts saliva and one or more oral surfaces during oral cavity exposure that provides modulation of an oral microenvironment, and wherein said composition subsequently undergoes gastrointestinal transit to support modulation of gut microbiome composition.

13. The composition of claim 1, wherein said pomegranate pomace serves dual functions within said composition, providing both antioxidant protection to said lipid phase and physical scaffolding for said biotic component.

14. The composition of claim 1, wherein said lipid phase comprises a combination of cocoa butter and mango butter in a ratio that is specifically configured to enable high loading of said biotic component and said pomegranate pomace and allows stabilization of crystallization in solid forms and/or causes phase separation in non-solid forms.

15. A functional food product, comprising:
a matrix that is a delivery system for bioactive compounds, said matrix comprising:
a lipid phase comprising one or more plant-derived lipids;
an antioxidant-rich botanical phase comprising one or more botanical materials homogeneously dispersed within said lipid phase; and
a biotic component comprising one or more of prebiotics, probiotics, postbiotics, and low-glycemic sweeteners homogeneously dispersed within said lipid phase in an absence of microencapsulation;
wherein said matrix is formulated in a solid and/or non-solid form and provides antioxidant activity, microbiome modulation, oral wellness support, and delivery of said bioactive compounds.

16. The functional food product of claim 15, wherein said plant-derived lipids are selected from at least one or more of a group consisting of cocoa butter, mango butter, shea butter, cocoa butter equivalents, medium chain triglycerides oil, and coconut oil.

17. The functional food product of claim 15, wherein said botanical materials are selected from at least one or more of a group consisting of cacao-derived materials, pomegranate pomace, carob, and mesquite.

18. The functional food product of claim 15, wherein said solid and/or non-solid form is selected from one or more of a group consisting of syrups, gels, pastes, spreads, and direct-to-mouth compositions.

19. The functional food product of claim 15, wherein said lipid phase comprises cocoa butter in a proportion of at least 20% by weight of said total lipid phase.

20. The functional food product of claim 15, wherein said antioxidant-rich botanical phase comprises a combination of cocoa liquor and pomegranate pomace, and further comprises carob and/or mesquite.

21. The functional food product of claim 15, wherein said biotic component comprises a combination of allulose as a prebiotic and one or more probiotic microorganisms.

22. The functional food product of claim 15, wherein said matrix exhibits a viscosity that enables dispensing from a squeeze pouch or tube at room temperature.

23. The functional food product of claim 15, wherein said matrix maintains physical stability in an absence of phase separation for at least six months at room temperature storage conditions.

24. The functional food product of claim 15, wherein said matrix is configured to function as a biphasic delivery system that releases a first bioactive portion comprising tagatose and polyphenols within an oral cavity during consumption and protects a second bioactive portion comprising said biotic component during gastric transit for colonic delivery.

25. A portion-controlled delivery system for bioactive compounds, comprising:
a sealed package that contains a pre-measured quantity of a matrix composition, said composition comprising:
a lipid phase comprising one or more plant-derived lipids selected from at least one or more of a group consisting of cocoa butter, mango butter, shea butter, cocoa butter equivalents, medium chain triglycerides oil and coconut oil;
an antioxidant-rich botanical phase comprising one or more of a group consisting of cacao-derived materials, pomegranate pomace, carob, and mesquite homogeneously dispersed within said lipid phase; and
a biotic component comprising one or more of prebiotics, probiotics, postbiotics, and low-glycemic sweeteners homogeneously dispersed within said lipid phase in an absence of microencapsulation;
wherein said sealed package is configured for direct dispensing of said composition into an oral cavity or onto food products.

26. The portion-controlled delivery system of claim 25, wherein said sealed package is a stick-pack.

27. The portion-controlled delivery system of claim 25, wherein said sealed package is a squeeze pouch.

28. The portion-controlled delivery system of claim 25, wherein said composition further comprises one or more natural sweeteners selected from one or more of a group consisting of monk fruit extract and stevia extract.

29. The portion-controlled delivery system of claim 25, wherein said composition further comprises one or more flavors selected from one or more of a group consisting of vanilla, mint, caramel, caramel cream, fruit, floral, citrus, and berry flavors, wherein said composition is formulated as a clean-label product that does not include synthetic encapsulating agents.

30. A form-agnostic food matrix composition, comprising:
a lipid phase comprising one or more plant-derived lipids;
an antioxidant-rich botanical phase comprising at least one or more of a group consisting of cacao-derived materials, pomegranate pomace, carob, and mesquite homogeneously dispersed within said lipid phase; and;
a biotic component comprising one or more of prebiotics, probiotics, postbiotics, and low-glycemic sweeteners homogeneously dispersed within said lipid phase in an absence of microencapsulation;
wherein said pomegranate pomace fibers dispersed within said lipid phase act as a physical scaffold that maintains matrix integrity in both solid and/or non-solid states, and wherein said composition maintains antioxidant and biotic viability regardless of physical form.
